(19) United States
(12) Patent Application Publication
(10) Pub. No.: US 2021/0290527 A1
(43) Pub. Date: Sep. 23, 2021

(54) Compositions and Methods for Treatment of Ocular Conditions

(71) Applicant: EyeGate Pharmaceuticals, Inc., Waltham, MA (US)

(72) Inventors: Brenda K. Mann, Salt Lake City, UT (US); Hee-Kyoung Lee, Salt Lake City, UT (US)

(73) Assignee: EyeGate Pharmaceuticals, Inc., Waltham, MA (US)

(21) Appl. No.: 17/208,381

(22) Filed: Mar. 22, 2021

Related U.S. Application Data
(60) Provisional application No. 63/048,936, filed on Jul. 7, 2020.
(60) Provisional application No. 62/993,384, filed on Mar. 23, 2020.

(51) Int. Cl. A61K 9/00; A61K 9/06; A61K 47/36; A61K 38/13; A61K 31/573

(52) U.S. Cl. CPC A61K 9/0048; A61K 9/06; A61K 31/573; A61K 38/13; A61K 47/36

(57) ABSTRACT

The present disclosure relates to compositions and methods for treating ocular conditions or diseases with low or poorly water-soluble therapeutics incorporated into a hydrogel. In particular, the disclosure relates to non-blurring, therapeutic-containing hydrogel compositions that have an extended contact time on the eye and do not interfere with wound healing.

### CROSS REFERENCE TO RELATED APPLICATIONS

This application claims the benefit of U.S. Provisional Application No. 62/993,384, filed Mar. 23, 2020, and U.S. Provisional Application No. 63/048,936, filed Jul. 7, 2020, both entitled “Compositions and Methods for Treatment of Ocular Diseases,” the entire contents of which are incorporated herein by reference.

### FIELD OF THE DISCLOSURE

The disclosure relates to compositions and methods for treating ocular conditions or diseases with therapeutics that are poorly water soluble and are incorporated beyond their solubility limit. In particular, the disclosure relates to non-blurring, therapeutic-containing hydrogel compositions that have an extended contact time on the eye and do not interfere with wound healing.

### BACKGROUND OF THE DISCLOSURE

Topical ophthalmic therapeutics are often prescribed to treat a variety of ocular conditions as it is a simple delivery method. However, many of these therapeutics have poor water solubility, and the desired concentration of the therapeutic in a formulation is above the solubility limit, complicating formulation of them into a simple eye drop. Generally, such low or poorly water-soluble therapeutics for topical ophthalmic application are formulated as a suspension, emulsion, or ointment. Unfortunately, suspensions and emulsions do not remain in contact with the eye for more than a few minutes because they are rapidly removed from the eye via factors such as tear turnover and gravity, thereby reducing the amount of drug that can be taken up into the ocular tissues. Ointments may have a longer contact time, but are often associated with blurring that interferes with a patient's vision. Further, other components required to formulate the therapeutic into the suspension, emulsion, or ointment may interfere with ocular surface healing required in conjunction with the ocular disease being treated. Thus, there is a need for compositions and methods for treating ocular disease with low or poorly water-soluble therapeutics that have an extended contact time on the eye, are non-blurring, and may aid in ocular surface healing.

### SUMMARY OF THE DISCLOSURE

The present disclosure provides a hydrogel that may be formulated to contain low or poorly water-soluble therapeutics to provide enhanced treatment of an ocular condition or disease, while simultaneously having the properties of increasing contact time of the therapeutic with the surface of the eye and providing clear vision for a subject (i.e., not blurring a subject's vision). Additionally, the therapeutic-containing hydrogel disclosed herein also has the ability to aid in the wound healing process. The hydrogel is shear-thinning and comprises modified or unmodified hyaluronic acid that is covalently crosslinked.

In one aspect, the disclosure provides an ocular composition that includes a shear-thinning hydrogel and a therapeutic that has a solubility in water of less than about 1.5 mg/ml. In an embodiment, the shear-thinning hydrogel may include hyaluronic acid, which may be at a concentration of between about 3 mg/ml and about 10 mg/ml. In an embodiment, the hyaluronic acid may be covalently crosslinked.

In an embodiment, the hyaluronic acid is modified or unmodified hyaluronic acid.

In an embodiment, the therapeutic has a concentration in the composition at least about 10 times greater than the solubility in water.

In an embodiment, the therapeutic has a concentration in the composition at least about 100 times greater than the solubility in water.

In an embodiment, the therapeutic is an antibiotic.

In an embodiment, the therapeutic is an antimicrobial agent.

In an embodiment, the therapeutic is an antiviral agent.

In an embodiment, the therapeutic is an immunosuppressant.

In an embodiment, the therapeutic is cyclosporin.

In an embodiment, the therapeutic is an anti-inflammatory agent.

In an embodiment, the therapeutic is a corticosteroid.

In an embodiment, the therapeutic is prednisolone or a salt of prednisolone.

In an embodiment, the therapeutic is an antihistamine.

In an embodiment, the modified hyaluronic acid is thiolated hyaluronic acid.

In an embodiment, the modified hyaluronic acid is thiolated carboxymethyl hyaluronic acid.

In an embodiment, the hydrogel may be disulfide crosslinked.

In an aspect, the disclosure provides an ocular composition that includes a shear-thinning hydrogel and a therapeutic that has a solubility in water of less than about 1.5 mg/ml. In an embodiment, the hydrogel may include thiolated hyaluronic acid, which may be present at a concentration of between about 3 mg/ml and about 10 mg/ml. In an embodiment, the hyaluronic acid may be disulfide crosslinked. In an embodiment, the therapeutic may be at a concentration of at least about 10 times the solubility in water.

In an embodiment, the thiolated hyaluronic acid has a thiol modification of about 0.05 μmol to about 1.0 μmol thiol/mg.

In an embodiment, the thiol modification is about 0.05 μmol to about 0.2 μmol thiol/mg, and the thiolated hyaluronic acid is at a concentration of about 6.5 mg/ml to about 8.5 mg/ml.

In an embodiment, the therapeutic is an immunosuppressant.

In an embodiment, the therapeutic is an anti-inflammatory agent.

In an embodiment, the therapeutic is a corticosteroid.

In an embodiment, the therapeutic is prednisolone or a salt of prednisolone.

In an embodiment, the disclosure provides a method of treating ocular disease in a subject, which includes a step of applying any of the above-described compositions to an eye of a subject.

In embodiments, the ocular disease may include conjunctivitis, diabetic retinopathy, dry eye, eye infections, glaucoma, macular degeneration, ocular allergies, presbyopia, retinal detachment, or uveitis.

In embodiments, the therapeutic agent may have a solubility in water of less than about 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.01 mg/ml, or 0.001 mg/ml.

### Definitions

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). Unless otherwise clear from context, all numerical values provided herein are modified by the term “about.”

By “control” or “reference” is meant a standard of comparison. In one aspect, as used herein, “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. Determination of statistical significance is within the ability of those skilled in the art, e.g., the number of standard deviations from the mean that constitute a positive result.

Unless specifically stated or obvious from context, as used herein, the term “or” is understood to be inclusive. Unless specifically stated or obvious from context, as used herein, the terms “a”, “an”, and “the” are understood to be singular or plural.

As used herein, the term “shear-thinning” refers to a state in which viscosity decreases as shear rate increases, thereby indicating shear-thinning behavior.

As used herein, the term “subject” includes humans and mammals (e.g., mice, rats, pigs, cats, dogs, and horses). In many embodiments, subjects are mammals, particularly primates, especially humans. In some embodiments, subjects are livestock such as cattle, sheep, goats, cows, swine, and the like; poultry such as chickens, ducks, geese, turkeys, and the like; and domesticated animals particularly pets such as dogs and cats. In some embodiments (e.g., particularly in research contexts) subject mammals will be, for example, rodents (e.g., mice, rats, hamsters), rabbits, primates, or swine such as inbred pigs and the like.

As used herein, the terms “treatment,” “treating,” “treat” and the like, refer to obtaining a desired pharmacologic and/or physiologic effect (e.g., reducing or eliminating a bacterial infection). The effect can be prophylactic in terms of completely or partially preventing a disease or infection or symptom thereof and/or can be therapeutic in terms of a partial or complete cure for a disease or infection and/or adverse effect attributable to the disease or infection. “Treatment,” as used herein, covers any treatment of a disease or condition or infection (e.g., an ocular infection) in a mammal, particularly in a human, and includes: preventing the disease or infection from occurring in a subject which can be predisposed to the disease or infection but has not yet been diagnosed as having it, inhibiting the disease or infection (e.g., arresting its development), relieving the disease or infection, reducing or eliminating a bacterial infection, and the like.

The term “pharmaceutically acceptable salts, esters, amides, and prodrugs” as used herein refers to those carboxylate salts, amino acid addition salts, esters, amides, and prodrugs of the compounds of the present disclosure which are, within the scope of sound medical judgment, suitable for use in contact with the tissues of patients without undue toxicity, irritation, allergic response, and the like, commensurate with a reasonable benefit/risk ratio, and effective for their intended use, as well as the zwitterionic forms, where possible, of the compounds of the disclosure.

The term “salts” refers to the relatively non-toxic, inorganic and organic acid addition salts of compounds of the present disclosure. These salts can be prepared in situ during the final isolation and purification of the compounds or by separately reacting the purified compound in its free base form with a suitable organic or inorganic acid and isolating the salt thus formed. Representative salts include the hydrobromide, hydrochloride, sulfate, bisulfate, nitrate, acetate, oxalate, valerate, oleate, palmitate, stearate, laurate, borate, benzoate, lactate, phosphate, tosylate, citrate, maleate, fumarate, succinate, tartrate, naphthylate mesylate, glucoheptonate, lactobionate and laurylsulphonate salts, and the like. These may include cations based on the alkali and alkaline earth metals, such as sodium, lithium, potassium, calcium, magnesium, and the like, as well as non-toxic ammonium, tetramethylammonium, tetramethylammonium, methlyamine, dimethlyamine, trimethlyamine, triethlyamine, ethylamine, and the like. (See, for example, S. M. Barge et al., “Pharmaceutical Salts,” J. Pharm. Sci., 1977, 66:1-19 which is incorporated herein by reference). Salts that are known to be compatible with various ophthalmic therapeutics are specifically contemplated within the scope of the disclosure.

Ranges can be expressed herein as from “about” one particular value and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed. In this regard, ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 50 may comprise 1 to 10, 1 to 20, 1 to 30, and 1 to 40 in one direction, or 50 to 40, 50 to 30, 50 to 20, and 50 to 10 in the other direction.

A “therapeutically effective amount” of an agent described herein is an amount sufficient to provide a therapeutic benefit in the treatment of a condition or to delay or minimize one or more symptoms associated with the condition (e.g., an amount sufficient to reduce or eliminate a bacterial infection in an eye of a subject). A therapeutically effective amount of an agent means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

Where applicable or not specifically disclaimed, any one of the embodiments described herein are contemplated to be able to combine with any other one or more embodiments, even though the embodiments are described under different aspects of the disclosure.

Other features and advantages of the disclosure will be apparent from the following description of the preferred embodiments thereof, and from the claims. Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described below. All published foreign patents and patent applications cited herein are incorporated herein by reference. All other published references, documents, manuscripts and scientific literature cited herein are incorporated herein by reference. In addition, the materials, methods, and examples are illustrative only and not intended to be limiting.

These and other embodiments are disclosed and/or encompassed by, the following detailed description.

### BRIEF DESCRIPTION OF THE DRAWINGS

The following detailed description, given by way of example, but not intended to limit the disclosure solely to the specific embodiments described, may best be understood in conjunction with the accompanying drawings, in which:

FIGS. 1A and 1B are graphs showing viscosity as a function of shear rate for hydrogel (“OBG”) alone (black squares) or with therapeutic incorporated. FIG. 1A shows the viscosity for prednisolone acetate (PredA) incorporated at a concentration of 1 (red circles), 5 (green circles), or 10 (blue circles) mg/ml. FIG. 1B shows the viscosity for loteprednol etabonate (LE) incorporated at a concentration of 1 (red circles), 2.5 (green circles), or 5 (blue circles) mg/ml.

FIGS. 2A and 2B are graphs showing viscosity as a function of shear rate for hydrogel (“OBG”) alone (black squares) or with therapeutic incorporated at a concentration of 1 mg/ml. FIG. 1A shows the viscosity for olopatadine (Olo) incorporated into the hydrogel (green triangles). FIG. 1B shows the viscosity for dexamethasone (Dex) incorporated into the hydrogel (red circles).

FIGS. 3A, 3B, 3C, and 3D show the release of therapeutic from hydrogel (“OBG”; blue circles on the graphs) or PBS (green circles on the graphs) into PBS in a medi-dialysis chamber (MWCO, 50 kDa) over time. FIG. 3A shows the release of PredA incorporated at 2 mg/ml. FIG. 3B shows the release of LE incorporated at 1 mg/ml. FIG. 3C shows the release of Olo incorporated at 1 mg/ml. FIG. 3D shows the release of Dex incorporated at 1 mg/ml.

### DETAILED DESCRIPTION OF THE DISCLOSURE

The present disclosure is based, at least in part, on the discovery that a hydrogel may be formulated to contain low or poorly water-soluble therapeutics (e.g., a water solubility of less than about 1.5 mg/ml) to provide enhanced prevention/treatment of ocular diseases, while simultaneously having increased contact time with the surface of the eye in a way that allows the subject to have clear vision (e.g., does not blur a subject's vision). The present disclosure provides therapeutic-containing hydrogels formulated in an exemplary embodiment as an eye drop. Advantageously, the therapeutic-containing hydrogel is formulated to be non-blurring while having extended contact time with the surface of the eye, which provides beneficial effects in terms of preventing or treating an ocular disease. Moreover, the therapeutic-containing hydrogel disclosed herein also has the ability to aid in the wound healing process. The hydrogel is shear-thinning and comprises modified or unmodified hyaluronic acid that is covalently crosslinked. In one aspect, the therapeutic has a solubility in water of less than about 1.5 mg/ml and is at a concentration of at least about 10 times the water solubility. It is contemplated within the scope of the disclosure that the therapeutic may have a solubility in water that is between about 0.001 mg/ml and 0.05 mg/ml, or between about 0.05 mg/ml and about 0.5 mg/ml, or between about 0.5 mg/ml and about 1.0 mg/ml, or between about 1.0 mg/ml and about 1.5 mg/ml. In some embodiments, the therapeutic may have a solubility in water that is less than about 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.01 mg/ml, or 0.001 mg/ml. In another aspect, the therapeutic is a corticosteroid. The compositions herein provide a number of advantages over the prior art, including: enhanced residence time on the surface of the eye, a non-blurring ophthalmic formulation, and the ability to deliver a poorly water-soluble therapeutic from a hydrogel.

### Compounds of the Disclosure

An ocular composition in the form of a hydrogel is provided that incorporates a low or poorly water-soluble therapeutic. The hydrogel is a covalently crosslinked hyaluronic acid (HA), and the hyaluronic acid may be modified or unmodified. Unmodified hyaluronic acid may be covalently crosslinked by a variety of methods, including crosslinking using 1,4-butanediol diglycidyl ether (BDDE), divinylsulfone, and dihydrazide. The hyaluronic acid may be modified to change the charge of the molecule, change its biological activity, or to include groups that may be used for crosslinking purposes. Particularly useful are thiolated hyaluronic acid or thiolated carboxymethyl hyaluronic acid (CMHA). Modified hyaluronic acid may be crosslinked with an external molecule for crosslinking, or without an external crosslinker molecule. For crosslinking thiolated HA or CMHA, a molecule with thiol-reactive sites, such as acrylates, methacrylates, haloacetates, haloacetamides, or maleimides, may be used as an external crosslinker molecule, examples of which include poly(ethylene glycol) diacrylate and poly(ethylene glycol) bisbromoacetate. For crosslinking without an external crosslinker molecule, in particular, thiolated HA or CMHA may be disulfide crosslinked via an oxidation process. Such disulfide crosslinking may be aided by use of an oxidant such as sodium hypochlorite or peroxide.

When modified HA is crosslinked via the modification (e.g., disulfide crosslinking of thiolated HA), the level of modification may be adjusted to control the amount of crosslinking of the hydrogel, such that a higher level of modification leads to more crosslinking. Particularly useful for formulating hydrogels of the present disclosure is thiolated HA or thiolated CMHA, where the thiol modification is about 0.05 to about 1.0 μmol thiol per mg of HA or CMHA. Modification levels within this range are particularly suitable for forming crosslinked hydrogels with a desired shear-thinning profile and viscosity.

When placed on the surface of the eye, shear-thinning hydrogels made using thiolated CMHA and having a concentration range of about 3 to about 10 mg/ml remain in contact with the eye surface for at least 30 minutes and up to about 2 hours.

### Combination Treatments

The therapeutic-containing hydrogel compositions and methods described herein may be used to direct the administration of combination therapies to treat particular ocular diseases. In order to increase the effectiveness of a treatment with the compositions of the present disclosure, e.g., an antibiotic selected and/or administered as a single agent, or to augment the protection of another therapy (second therapy), it may be desirable to combine these compositions (e.g., include more than one therapeutic in the therapeutic-containing hydrogel compositions) and methods with one another, or with other agents and methods effective in the treatment, amelioration, or prevention of diseases and pathologic conditions.

Administration of a composition of the present disclosure to a subject will follow general protocols for the administration described herein, and the general protocols for the administration of a particular secondary therapy will also be followed, taking into account the toxicity, if any, of the treatment. It is expected that the treatment cycles would be repeated as necessary.

### Pharmaceutical Compositions

Therapeutics that may be incorporated in the therapeutic-containing hydrogel compositions disclosed herein are those that are clinically relevant for ocular conditions and may include antimicrobials, antibiotics, antiviral agents, immunosuppressants, anti-inflammatory agents, antihistamines, and combinations thereof. Particularly useful in the present disclosure are therapeutics that have a solubility in water of less than about 1.5 mg/ml. It is contemplated within the scope of the disclosure that the therapeutic may have a solubility in water that is between about 0.001 mg/ml and 0.05 mg/ml, or between about 0.05 mg/ml and about 0.5 mg/ml, or between about 0.5 mg/ml and about 1.0 mg/ml, or between about 1.0 mg/ml and about 1.5 mg/ml. In some embodiments, the therapeutic may have a solubility in water that is less than about 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.01 mg/ml, or 0.001 mg/ml. An exemplary immunosuppressant that may be used in cyclosporin (water solubility <0.05 mg/ml). An exemplary anti-inflammatory agent that may be used is a corticosteroid, e.g., prednisolone acetate (water solubility <0.05 mg/ml). An exemplary antiviral agent that may be used is acyclovir (water solubility of 1.4 mg/ml). The therapeutic may be incorporated at a concentration of at least about 10 to 100 times the water solubility of the therapeutic, and may be incorporated prior to, during, or after crosslinking of the hydrogel.

### Method of Treatment

The topical application of a therapeutic can be used to treat or prevent a variety of ocular conditions or diseases including bacterial or viral infections, post-surgical pain, inflammation, correctable congenital defects, and allergic reactions. Ocular conditions or diseases which may benefit from therapeutic application of the present invention include but are not limited to: conjunctivitis, diabetic retinopathy, dry eye, eye infections, glaucoma, macular degeneration, ocular allergies, presbyopia, retinal detachment, and uveitis.

The agents contained in the disclosed drug delivery systems will be released from the therapeutic-containing hydrogel compositions at rates that depend on such factors as the therapeutic itself and its physical form and the concentration of therapeutic in the hydrogel.

The therapeutics used in the present invention are commercially available or readily obtained by a worker skilled in the art through known reactions techniques. The therapeutic can be combined with the other ingredients in the chosen dosage form by conventional methods known in the art.

The therapeutic-containing hydrogel composition is topically applied to an eye of a human or non-human animal, the latter including cows, sheep, horses, pigs, goats, rabbits, dogs, cats, and other mammals. The composition can be topically applied, without limitation, to the front of the eye, under the upper eyelid, on the lower eyelid and in the cul-de-sac. The application can be as a treatment of a condition or disease of the eye or as a preventive such as prior to surgery.

### Kits

In general, therapeutic-containing hydrogel compositions of the invention may be provided as a kit that contains the therapeutic or compositions of the invention packaged to facilitate dispensing and/or applying the composition to affected or susceptible regions of the eye. The packaging or dispenser may include a dropper, bottle, tube, spray bottle, or other dispenser and instructions for use.

The kits are manufactured using medically acceptable conditions and contain components that have sterility, purity and preparation that is pharmaceutically acceptable.

The instructions generally include information as to dosage, dosing schedule, and route of administration for the intended treatment. The containers may be unit doses, bulk packages (e.g., multi-dose packages) or sub-unit doses. Instructions supplied in the kits of the instant disclosure are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating or preventing an ocular condition or disease in a subject. Instructions may be provided for practicing any of the methods described herein.

The kits of this disclosure are in suitable packaging. Suitable packaging includes, but is not limited to, droppers, vials, bottles, jars, flexible packaging (e.g., sealed Mylar or plastic bags), and the like. The container may further comprise a second pharmaceutically active agent.

Kits may optionally provide additional components such as buffers and interpretive information. Normally, the kit comprises a container and a label or package insert(s) on or associated with the container.

Reference will now be made in detail to exemplary embodiments of the disclosure. While the disclosure will be described in conjunction with the exemplary embodiments, it will be understood that it is not intended to limit the disclosure to those embodiments. To the contrary, it is intended to cover alternatives, modifications, and equivalents as may be included within the spirit and scope of the disclosure as defined by the appended claims.

### Examples

The present disclosure is further illustrated by the following examples, which should not be construed as limiting. The contents of all references, published patents and patent applications cited throughout the application are hereby incorporated by reference. Those skilled in the art will recognize that the disclosure may be practiced with variations on the disclosed structures, materials, compositions and methods, and such variations are regarded as within the scope of the disclosure.

### Example 1: Hydrogel Formation

Thiol-modified carboxymethyl HA (CMHA-S) was synthesized as described in Lawyer et al. [1] and Wendling et al. [2], with a thiol modification of 0.1, 0.2, 0.4, or 0.7 μmol thiol/mg. Hydrogels were created by dissolving CMHA-S in phosphate-buffered saline (PBS; pH 7.4). The CMHA-S was disulfide crosslinked under continuous mixing with the addition of sodium hypochlorite. Rheological testing was performed using a parallel plate format rheometer with a 25 mm-diameter stainless steel geometry. Samples (5-6 ml) of hydrogel were placed in a 35 mm Petri dish, and the geometry was lowered to a gap of 5 mm. To determine viscosity and shear-thinning, the shear rate was varied from 0.1 to 10 Hz. A decreasing viscosity as shear rate increases indicates shear-thinning behavior. Table 1 provides the thiol modification of the CMHA-S, concentration of CMHA-S, and resultant viscosity of the hydrogel (at 2.5 Hz) for 4 hydrogel formulations. All 4 formulations displayed shear-thinning behavior.

TABLE 1
Examples of hydrogel formulations and viscosity.

| | Thiol | CMHA-S | |
|---|---|---|---|
| | modification | concentration | Viscosity at |
| Hydrogel # | (μmol thiol/mg) | (mg/ml) | 2.5 Hz (Pa · s) |
| 1 | 0.12 | 8.3 | 2.6 |
| 2 | 0.15 | 7.8 | 3.6 |
| 3 | 0.13 | 7.3 | 2.4 |
| 4 | 0.39 | 4.0 | 0.9 |

### Example 2: Therapeutic-Containing Hydrogels

Therapeutics were mixed into a hydrogel made as in Example 1, with a thiol modification about 0.1 μmol thiol/mg and CMHA-S concentration about 7.5 mg/ml. Therapeutics used were: prednisolone acetate (predA) at a concentration of 1, 5, or 10 mg/ml; loteprednol etabonate (LE) at a concentration of 1, 2.5, or 5 mg/ml; and olopatadine (Olo) and dexamethasone (Dex), each at a concentration of 1 mg/ml. PredA is considered poorly soluble to practically insoluble in water. LE has a water solubility of less than 0.001 mg/ml. Olo has a water solubility of about 0.03 mg/ml. Dex has a water solubility of less than 0.09 mg/ml. All of these therapeutics are therefore considered to have very low to poor solubility in water. Therapeutics were added as a finely ground powder to the crosslinked hydrogel and the mixture stirred or shaken vigorously to incorporate the therapeutic throughout. The therapeutic was dispersed throughout the hydrogel but was not fully dissolved.

### Example 3: Physical Properties of Therapeutic-Containing Hydrogels

Viscosity, pH, and refractive index (RI) were measured for hydrogels described in Example 2 with and without therapeutic incorporated. Viscosity was determined as described in Example 1. RI was measured with a refractometer and pH was measured with a pH meter.

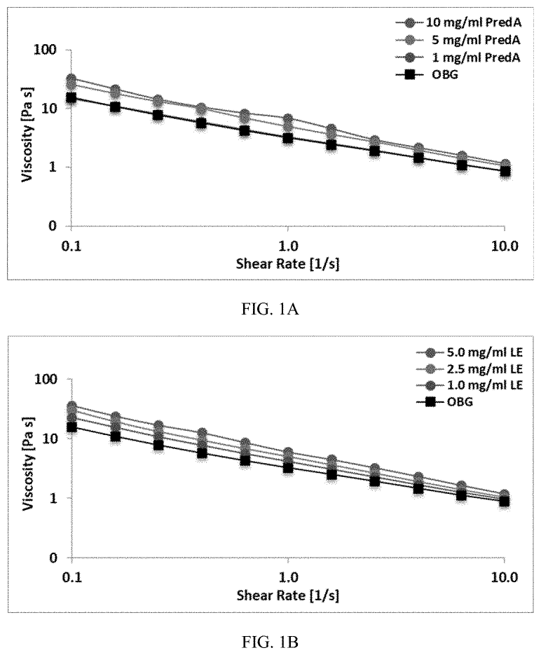

For predA, increasing concentration of the therapeutic in the hydrogel led to a slight increase in the viscosity (Table 2), although not significantly different than hydrogel without the predA, and the hydrogel maintained its shear thinning property (FIG. 1A). Further, the addition of the therapeutic did not substantially change the refractive index or pH of the hydrogel. The hydrogel did become slightly more opaque with increasing concentration of the predA; however, because only a small drop would be used and would spread across the surface of the eye, it would not be considered to be blur the vision.

TABLE 2
Viscosity, refractive index (RI), and pH of hydrogels with
and without prednisolone acetate (predA) incorporated.

| PredA | | | |
|---|---|---|---|
| Concentration | Viscosity at | | |
| (mg/ml) | 2.5 Hz (Pa · s) | RI | pH |
| 0 | 1.9 | 1.3354 | 7.05 |
| 1 | 2.0 | 1.3354 | 7.09 |
| 5 | 2.7 | 1.3355 | 7.13 |
| 10 | 3.0 | 1.3354 | 7.17 |

Similar results were seen for LE incorporated into the hydrogels (FIG. 1B and Table 3). The pH increased a little more with increasing amounts of LE compared to predA, although the pH remained well within the acceptable range for topical ophthalmics (generally, a pH of about 6-8), and the pH could be adjusted either before or after incorporation of the drug if desired.

TABLE 3
Viscosity, refractive index (RI), and pH of hydrogels with
and without loteprednol etabonate (LE) incorporated.

| LE Concentration | Viscosity at | | |
|---|---|---|---|
| (mg/ml) | 2.5 Hz (Pa · s) | RI | pH |
| 0 | 1.9 | 1.3357 | 7.04 |
| 1 | 2.3 | 1.3355 | 7.11 |
| 2.5 | 2.7 | 1.3354 | 7.24 |
| 5 | 3.3 | 1.3353 | 7.55 |

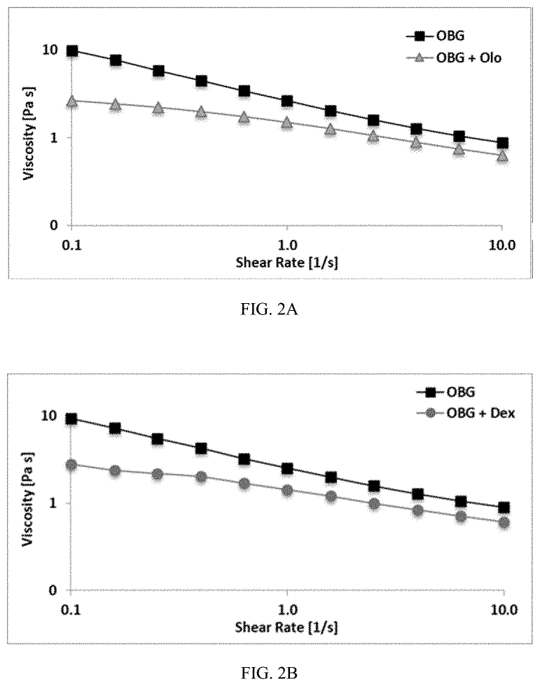

For olopatadine and dexamethasone, the physical appearance (transparency), pH, and refractive index of the hydrogel were the same with and without the therapeutic incorporated (Table 4), although the appearance was slightly more opaque with dexamethasone incorporated. The viscosity decreased compared to hydrogel without the therapeutic (Table 4), although the hydrogel with therapeutic still demonstrated shear-thinning properties (FIGS. 2A and 2B), and the viscosity was still within the desired specifications for the hydrogel. Further, a hydrogel with a slightly higher viscosity could be used for incorporating these therapeutics to ensure a final viscosity in a desired range.

TABLE 4
Viscosity, refractive index (RI), and pH of hydrogels
with and without olopatadine or dexamethasone
incorporated at a concentration of 1 mg/ml.

| | Viscosity at | | |
|---|---|---|---|
| Drug incorporated | 2.5 Hz (Pa · s) | RI | pH |
| None | 1.6 | 1.3353 | 7.3 |
| Olopatadine | 1.1 | 1.3353 | 7.3 |
| Dexamethasone | 1.0 | 1.3354 | 7.2 |

### Example 4: Release of Low/Poorly Soluble Therapeutics from Hydrogels

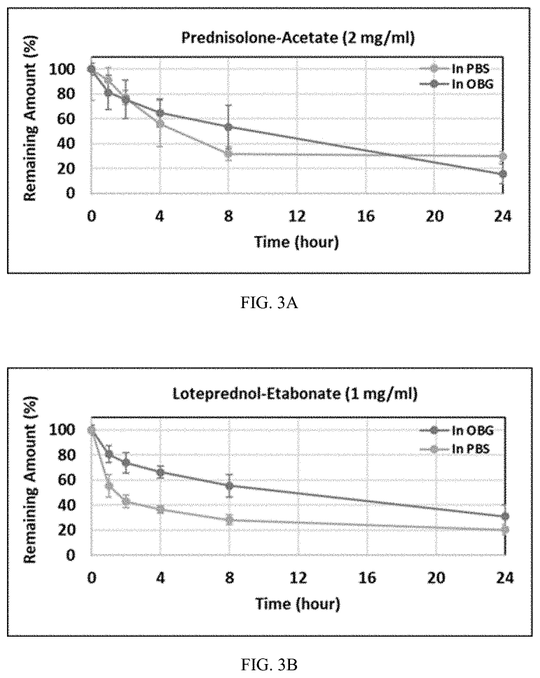

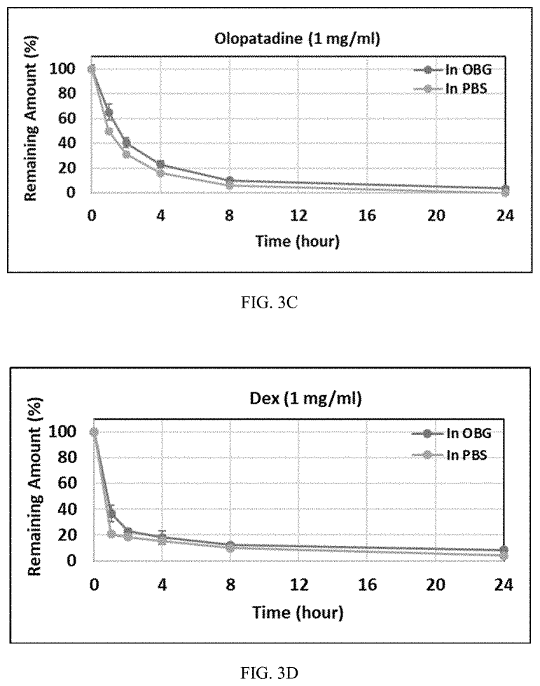

Hydrogels described in Example 2 containing PredA at 2 mg/ml, or LE, Olo, or Dex at 1 mg/ml, were used to assess the release of the therapeutic from the hydrogel. The release was monitored over 24 hours and compared to release of the therapeutic from solution in PBS. For this assessment, a 0.5 ml sample of hydrogel plus therapeutic or PBS plus therapeutic was placed into a medi-dialysis chamber, and the chamber was placed in a beaker containing 100 ml of PBS. The remaining amount of therapeutic in the dialysis chamber was monitored using UV spectroscopy at various time points. The release of PredA, olopatadine, and Dex from the hydrogel was similar to their release from PBS (FIGS. 3A, 3C, and 3D, respectively). Release of LE from the hydrogel was reduced compared to its release from PBS (FIG. 3B); however, release of the therapeutic still occurred, and such a reduction in the rate may lead to a more controlled delivery.

### INCORPORATION BY REFERENCE

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

### EQUIVALENTS

It is understood that the detailed examples and embodiments described herein are given by way of example for illustrative purposes only, and are in no way considered to be limiting to the disclosure. Various modifications or changes in light thereof will be suggested to persons skilled in the art and are included within the spirit and purview of this application and are considered within the scope of the appended claims. Additional advantageous features and functionalities associated with the systems, methods, and processes of the present disclosure will be apparent from the appended claims. Moreover, those skilled in the art will recognize, or be able to ascertain using no more than routine experimentation, many equivalents to the specific embodiments of the disclosure described herein. Such equivalents are intended to be encompassed by the following claims.

We claim:

1. An ocular composition, comprising:
a shear-thinning hydrogel comprising hyaluronic acid, wherein the hyaluronic acid is at a concentration of about 3 to about 10 mg/ml and is covalently crosslinked; and
a therapeutic agent, wherein the therapeutic agent has a solubility in water of less than about 1.5 mg/ml.

2. The ocular composition of claim 1, wherein the hyaluronic acid is modified or unmodified hyaluronic acid.

3. The ocular composition of claim 1, wherein the therapeutic agent has a concentration in the composition at least about 10 times greater than the solubility in water.

4. The ocular composition of claim 1, wherein the therapeutic agent has a concentration in the composition at least about 100 times greater than the solubility in water.

5. The ocular composition of claim 4, wherein the therapeutic agent comprises one or more of: an antimicrobial agent, an antibacterial agent, an antiviral agent, an immunosuppressant, an anti-inflammatory agent, and an antihistamine.

6. The ocular composition of claim 5, wherein the therapeutic is cyclosporin.

7. The ocular composition of claim 5, wherein the therapeutic is a corticosteroid.

8. The ocular composition of claim 7, wherein the therapeutic is prednisolone or a salt of prednisolone.

9. The ocular composition of claim 5, wherein the therapeutic is loteprednol or a salt of loteprednol.

10. The ocular composition of claim 2, wherein the modified hyaluronic acid is thiolated hyaluronic acid or thiolated carboxymethyl hyaluronic acid.

11. The composition of claim 10 wherein the hydrogel is disulfide crosslinked.

12. An ocular composition comprising:
a shear-thinning hydrogel comprising thiolated hyaluronic acid, wherein the thiolated hyaluronic acid is at a concentration of about 3 to about 10 mg/ml and is disulfide crosslinked; and
a therapeutic agent, wherein the therapeutic agent has a solubility in water of less than about 1.0 mg/ml, and the therapeutic agent is at a concentration in the composition of at least 10 times the solubility in water.

13. The composition of claim 12, wherein the thiolated hyaluronic acid has a thiol modification of about 0.05 μmol to about 1.0 μmol thiol/mg.

14. The composition of claim 13, wherein the thiol modification is about 0.05 μmol to about 0.2 μmol thiol/mg, and wherein the thiolated hyaluronic acid is at a concentration of about 6.5 mg/ml to about 8.5 mg/ml.

15. The composition of claim 12, wherein the therapeutic is a corticosteroid, optionally wherein the corticosteroid is prednisolone or a salt of prednisolone.

16. A method of treating ocular disease in a subject, comprising applying the composition of claim 1 to an eye of the subject.

17. The method of claim 16, wherein the ocular disease is selected from the group consisting of conjunctivitis, diabetic retinopathy, dry eye, eye infections, glaucoma, macular degeneration, ocular allergies, presbyopia, retinal detachment, and uveitis.

18. A method of treating ocular disease in a subject, comprising applying the composition of claim 12 to an eye of the subject.

19. The method of claim 18, wherein the ocular disease is selected from the group consisting of conjunctivitis, diabetic retinopathy, dry eye, eye infections, glaucoma, macular degeneration, ocular allergies, presbyopia, retinal detachment, and uveitis.

20. The ocular composition of claim 1, wherein the therapeutic agent has a solubility in water of less than about 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.01 mg/ml, or 0.001 mg/ml.
